(19) United States
(12) Patent Application Publication
(10) Pub. No.: US 2010/0323401 A1
(43) Pub. Date: Dec. 23, 2010

(54) GENERAL METHOD FOR GENERATING HUMAN ANTIBODY RESPONSES IN VITRO

(76) Inventors: George K. Lewis, Baltimore, MD (US); Yongjun Guan, Abingdon, MD (US)

Correspondence Address: MOORE & VAN ALLEN PLLC, P.O. BOX 13706, Research Triangle Park, NC, 27709, US

(21) Appl. No.: 12/300,461

(22) PCT Filed: May 11, 2007
(86) PCT No.: PCT/US07/68752

§ 371 Date: Apr. 6, 2009

Related U.S. Application Data
(60) Provisional application No. 60/747,021, filed on May 11, 2006.

(51) Int. Cl. C12P 21/08; G01N 33/50; C12N 5/0786; C12N 5/078; C12N 5/0783

(52) U.S. Cl. 435/71.1; 435/7.24; 435/404; 435/406

(57) ABSTRACT

This invention relates to an in vitro method of producing antigen specific B cells and antibodies that provides for the capture of an entire primary human antibody repertoire for any foreign antigen, allows for screening large numbers of immunogen/adjuvant combinations, and permits the isolation of human monoclonal antibodies on demand thereby obviating the need to immunize humans with the target antigen.

### CROSS REFERENCE TO RELATED APPLICATIONS

The present application claims priority to U.S. Provisional Patent Application No. 60/747,021 the contents of which are hereby incorporated by reference herein.

### STATEMENT OF GOVERNMENT RIGHTS

This work was supported by a grant from the National Institute of Health AIAI43046 and the United States Government may have right to this invention.

### BACKGROUND THE INVENTION

1. Field of the Invention

This invention relates generally to production of antigen specific B-Cells and antibodies, and more specifically, to an in vitro method of producing antibodies that provides for the capture of an entire primary human antibody repertoire for any foreign antigen, allows for screening large numbers of immunogen/adjuvant combinations as a prelude to Phase I vaccine trials, and permits the isolation of human monoclonal antibodies on demand thereby obviating the need to immunize humans with the target antigen.

2. Description of Related Art

Monoclonal antibodies derived from mice are the reagents most commonly used in in vivo therapeutic and diagnostic procedures and for in vitro diagnostic testing. Although a good deal of success has been had with the use of these murine monoclonals, a major disadvantage is that they are not identical to antibodies produced by humans. Because of the species differences, when use is made of murine monoclonal antibodies in the in vivo diagnostic or therapeutic treatment of humans, it is now known that anti-murine antibodies may be produced in the treated patient. In order to eliminate these problems, the monoclonal antibody of choice, especially for use in vivo, is usually derived from humans.

Notably, developing human monoclonal antibodies requires that the B cell donor be immune to the test antigen, and as such, immune individuals must be located as a source of B cells to generate monoclonal antibodies. However, time frames for locating or inducing an immune B cell donor is not possible if the target disease is rare or dangerous such as when dealing with ricin and botulinum toxin.

Although it is theoretically possible to produce human antibodies using cells from various organs, such as the spleen or the tonsils, the most readily available source is the peripheral blood supply. It is known to those skilled in the art that it is possible to produce antigen specific monoclonal antibodies derived from human peripheral blood lymphocytes (PBL). Further, it is well known that presentation of antigen in vitro is technically difficult and the success rate for producing antigen specific human antibodies is particularly time consuming.

To date, the only way to deliberately generate primary human antibody responses is to immunize a volunteer with an antigen or to follow antibody responses of an individual who becomes infected with a virus, bacterium, or parasite. This greatly complicates vaccine development as the only way to evaluate immunogenicity in humans is via clinical trials, which are expensive and time consuming. In addition, the need for in vivo immunization is a major impediment to the development of human monoclonal antibodies for passive immunotherapy, which is a rapidly growing segment of the pharmaceutical industry (1, 2).

With increased interest in human monoclonal antibodies, it has become necessary to devise ways of immunizing human lymphocytes in-vitro, however, heretofore the available systems can cause a decrease in antigen presenting cell, do not use cells populations from a single donor or use of naïve cells is lacking. Thus, there is an acute need for an in vitro system that allows the generation of human antibody responses to antigens on demand.

### SUMMARY OF THE INVENTION

The present invention relates to a system and method of generating primary antibodies in vitro in response to an antigen. This system can be used to generate and quantify primary antibody responses in humans. In addition to being useful for evaluating potential vaccines for human use, this system can be used to generate human antigen-specific B-cells for use in generating monoclonal antibodies on demand.

In one aspect, the present invention relates to a method of inducing the production of antigen specific B-cells, the method comprising the steps of:
(a) providing monocytes;
(b) differentiating the monocytes into monocyte derived dendritic cells;
(c) culturing the differentiated monocyte derived dendritic cells with isolated naive T and B cells in combination with a target antigen; and
(d) separating antigen specific B-cells.

In another aspect, the present invention relates to a two phase culturing method for generating antigen-specific B-cells and antibodies specific for the antigen, the method comprising:
(a) removing a blood sample from a donor;
(b) isolating naive T cells, naive B cells and monocytes from the sample into three separate populations;
(c) culturing the monocytes in a carrier for differentiating the monocytes into monocyte derived dendritic cells;
(d) culturing the monocytes derived dendritic cells with the naïve T cell, naïve B cells and the target antigen; and
(e) recovering generated antibodies.

In yet another aspect, the present invention relates to method for testing immunogen/adjuvant combinations to determine optimal level of antibody generation and/or most effective adjuvant, the method comprising:
(a) removing a blood sample from a donor;
(b) isolating naive T cells, naive B cells and monocytes from the sample into three separate populations;
(c) culturing the monocytes in a first culture medium for differentiating the monocytes into monocyte derived dendritic cells (MDC);
(d) isolating the monocyte derived dendritic cells from the first culture medium;
(e) culturing the monocytes derived dendritic cells with the naïve T cell, naïve B cells and the target antigen in a second culture medium;
(f) adding an adjuvant to the culture medium; and
(g) isolating recovered generated antibodies and determining level of immune response relative to a system without the addition of the adjuvant.

This process may be repeated numerous times with different adjuvants to determine the most effective adjuvant in inducing an immune response.

In a still further aspect, the present invention relates to the in vitro production of monoclonal antibodies without the need of a cell donor immune to the test antigen, the method comprising:
(a) removing a blood sample from a donor;
(b) isolating at least naive T cells, naive B cells and monocytes from the sample;
(c) differentiating the monocytes into monocyte derived dendritic cells;
(d) culturing the monocytes derived dendritic cells with T cell, B cells and the target antigen to generate antibody producing B cells;
(e) isolating the antibody producing B-cells;
(f) infecting the antibody producing B cells with Epstein Barr virus and/or fusing the antibody producing B cells with an active myeloma cell to form hybridoma cells;
(g) culturing the hybridoma cells under suitable conditions for expressing desired antibodies; and
(h) recovering the hybridoma cells that generate the desired monoclonal antibody and cloning same.

Another aspect of the present invention, relates to a two separate culture system for the in vitro production of antibodies, the system comprising:
(a) a first culture medium comprising monocytes isolated from sample and a activating agent for differentiating the monocyte into monocyte derived dendritic cells (MDCs); and
(b) a second culture medium comprising the MDC isolated from the first culture medium, naïve T cells and B cells isolated from the same sample as (a), a target antigen, and culturing components for the induction of antibodies.

A still further aspect of the present invention relates to a method for amplifying production of antigen specific human IgM, IgG and/or IgA monoclonal antibodies comprising:
(a) obtaining a human peripheral blood lymphocytes (PBL),
(b) separating naive B-cells and naive T-cells from said PBL,
(c) obtaining monocytes and differentiating the monocytes in the presence of an activating agent to form MDCs;
(d) combining the MDC, naïve T-cells and naïve B-cells in the presence of an antigen and optionally an adjuvant;
(e) identifying cells producing antigen specific IgM antibodies, and
(f) cloning said identified cells.

Other aspects and advantages of the invention will be more fully apparent from the ensuing disclosure and appended claims

### BRIEF DESCRIPTION OF THE FIGURES

FIG. 1 is a schematic for production of antibodies using the method of the present invention.

FIG. 2 illustrates the response of the highly fluorescent protein, allophycocyanin (APC (5)) with generated anti-APC antibodies.

### DETAILED DESCRIPTION OF THE PRESENT INVENTION

Due to advances in the understanding of cellular cooperation in the generation of antibody responses (3) and in the ability to rapidly purify the relevant cells from human peripheral blood, a system has been developed to generate primary human antibody responses on demand. Accordingly, the present invention provides for a system and method that can be utilized in determining the effectiveness of a candidate vaccine in the induction of an immune response including antibody production, thereby eliminating the need for use of animal models, such as murine models, that are frequently not predictive of the responses obtained in humans.

The practice of the present invention will employ, unless otherwise indicated, conventional techniques of immunology, molecular biology, microbiology, cell biology and recombinant DNA, which are within the skill of the art. See, e.g., Sambrook, et al. MOLECULAR CLONING: A LABORATORY MANUAL, 2nd edition (1989); CURRENT PROTOCOLS 1N MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

As used in the specification and claims, the singular form “a”, “an” and “the” include plural references unless the context clearly dictates otherwise. For example, the term “a cell” includes a plurality of cells, including mixtures thereof.

The terms “immunoglobulin molecule” or “antibodies,” as used herein, mean molecules that contain an antigen binding site which specifically binds an antigen. Structurally, the simplest naturally occurring antibody (e.g., IgG) comprises four polypeptide chains, two heavy (H) chains and two light (L) chains inter-connected by disulfide bonds. The natural immunoglobulins represent a large family of molecules that include several types of molecules, such as IgD, IgG, IgA, IgM and IgE.

The present invention may be used with any antigen which can be derived from multiple sources. The selection of an immunogen against which antibodies are to be raised will, of course, depend upon clinical interest. Some clinically significant immunogens include bacterial antigens, viral antigens, toxins, blood group antigens, antigens on lymphoid cells, myosin, and tumor antigens such as cell-associated antigens and tumor cell secreted products. As is well known in the art, smaller antigens (mol. wt. of less than about 5000) may need to be coupled to a carrier in order to stimulate an effective immune response against the immunogen. Preferably, this carrier is an entity against which the human lymphocytes have been previously immunized, e.g. tetanus toxoid. Examples of useful carriers are keyhole-limpet hemocyanin, thyroglobulin, albumins, muramylxdipeptide, red blood cells, a solid matrix such as Sepharose beads, alkaline phosphatase, globulins, synthetic copolymers, fibrinogen and the like.

Some smaller antigens may also be polymerized to increase immunogenicity. Linking agents useful in the coupling of smaller antigens to carriers include carbodiimides; glutaraldehyde; N-N-carbonyldiimidazole; 1-hydroxybenzotriazole monohydrate; N-hydroxy succinimide; N-trifluoroacetylimidazole; cyanogen bromide; and bis-diazotized benzidine.

The antigen may be either a foreign antigen or an endogenous antigen. As used herein, “foreign antigen” refers to a protein or fragment thereof, which is foreign to the recipient animal cell or tissue including, but not limited to, a viral protein, a parasite protein, or an immunoregulatory agent. The term “endogenous antigen” is used herein to refer to a protein or part thereof that is naturally present in the recipient animal cell or tissue, such as a cellular protein, or immunoregulatory agent.

Alternatively, the foreign antigen may be encoded by a synthetic gene and may be constructed using conventional recombinant DNA or RNA methods (See example 1 for synthetic gene construction procedures); the synthetic gene may express antigens or parts thereof that originate from viral and parasitic pathogens. These pathogens can be infectious in humans, domestic animals or wild animal hosts.

The foreign antigen can be any molecule that is expressed by any viral or parasitic pathogen prior to or during entry into, colonization of, or replication in their animal host.

Viral pathogens, from which viral antigens are derived may include, but are not limited to, Orthomyxoviruses, such as influenza virus; Retroviruses, such as RSV, HTLV-1 and HTLV-II; Herpes viruses, such as EBV, CMV or herpes simplex virus; Lentiviruses, such as HIV-1 and HIV-2; Rhabdoviruses, such as rabies; Picornoviruses, such as Poliovirus; Poxviruses, such as vaccinia; Rotavirus; Rhinovirus and Parvoviruses, such as adeno-associated virus 1 (AAV-1).

Examples of viral antigens include, but are not limited to, the human immunodeficiency virus antigens Nef, Gag, Env, Tat, Rev, Pol and T cell and B cell epitopes of gp120, such as CD4, fragment thereof or mimetics thereof; chimeric polypeptides including receptor-ligand pairs including env proteins and virus antigens, such as VP4 and VP7; influenza virus antigens, such as hemagglutinin; nucleoprotein; herpes simplex virus antigens; and toxins such as botulism, spider toxins; hepatitis B surface antigen; other toxins including avian viruses

The bacterial pathogens, from which the bacterial antigens are derived, include but are not limited to, Mycobacterium spp., Helicobacter pylori, Salmonella spp., Shigella spp., E. coli, Rickettsia spp., Listeria spp., Legionella pneumoniae, Pseudomonas spp., Vibrio spp., and Borellia burgdorferi.

Examples of protective antigens of bacterial pathogens include the somatic antigens of enterotoxigenic E. coli, such as the CFA/I fimbrial antigen and the nontoxic B-subunit of the heat-labile toxin; pertactin of Bordetella pertussis, adenylate cyclase-hemolysin of B. pertussis, fragment C of tetanus toxin of Clostridium tetani, OspA of Borellia burgdorferi, protective paracrystalline-surface-layer proteins of Rickettsia prowazekii and Rickettsia typhi, the listeriolysin (also known as “Llo” and “Hly”) and/or the superoxide dismutase (also know as “SOD” and “p60”) of Listeria monocytogenes; the urease of Helicobacter pylori, and the receptor-binding domain of lethal toxin and/or the protective antigen of Bacillus anthrax.

Example of antigens from biological weapons or pathogens include, but are not limited to, smallpox, anthrax, tularemia, plague, listeria, brucellosis, hepatitis, vaccinia, mycobacteria, coxsackievirus, tuberculosis, malaria, erhlichosis and bacterial meningitis.

The parasitic pathogens, from which the parasitic antigens are derived, include but are not limited to, Plasmodium spp., such as Plasmodium falciparum; Trypanosome spp., such as Trypanosoma cruzi; Giardia spp., such as Giardia intestinalis; Boophilus spp.; Babesia spp., such as Babesia microti; Entamoeba spp., such as Entamoeba histolytica; Eimeria spp., such as Eimeria maxima; Leishmania spp., Schistosome spp., such as Schistosoma mansoni; Brugia spp., such as Brugia malayi; Fascida spp., such as Fasciola hepatica; Dirofilaria spp., such as Dirofilaria immitis; Wuchereria spp., such as Wuchereria bancrofti; and Onchocerea spp; such as Onchocerca volvulus.

Examples of parasite antigens include, but are not limited to, the pre-erythrocytic stage antigens of Plasmodium spp., such as the circumsporozoite antigen of P. falciparum; P vivax; the liver stage antigens of Plasmodium spp., such as the liver stage antigen 1; the merozoite stage antigens of Plasmodium spp., such as the merozoite surface antigen-1 (also referred to as MSA-1 or MSP-1); the surface antigens of Entamoeba histolytic, such as the galactose specific lectin or the serine rich Entamoeba histolytica protein (also referred to as SREHP); the surface proteins of Leishmania spp. (also referred to as gp63); such as 63 kDa glycoprotein (gp63) of Leishmania major or the 46 kDa glycoprotein (gp46) of Leishmania major; paramyosin of Brugia malayi; the triose-phosphate isomerase of Schistosoma mansoni; the secreted globin-like protein of Trichostrongylus colubriformis; the glutathione-S-transferases of Fasciola hepatica; Schistosoma bovis; S. japonicum; and KLH of Schistosoma bovis and S. japonicum.

Examples of tumor specific antigens include prostate specific antigen (PSA), TAG-72 and CEA; human tyrosinase; tyrosinase-related protein (also referred to as TRP); and tumor-specific peptide antigens.

Examples of transplant antigens include the CD3 molecule on T cells and histocompatibility antigens such as HLA A, HLA B, HLA C, HLA DR and HLA DQ.

Examples of autoimmune antigens include IAS β chain, which is useful in therapeutic vaccines against autoimmune encephalomyelitis; glatamic acid decarboxylase, which is useful in therapeutic vaccines against insulin-dependent type 1 diabetes; thyrotropin receptor (TSHr), which is useful in therapeutic vaccines against Grave's disease and tyrosinase-related protein 1, which is useful in therapeutic vaccines against vitiligo.

Endogenous antigen, which may be any cellular protein or immunoregulatory agent, or parts thereof, expressed in the blood donor are also applicable to the present invention including, but not limited to, tumor, transplantation and autoimmune antigens, or fragments and derivatives of tumor, transplantation and autoimmune antigens thereof.

The concentration of the antigen necessary for purposes of the instant invention will depend upon the size of the antigen, and will generally be in the range of 0.1 to 100,000 ng/ml, preferably 1-10,000 ng/ml.

In the practice of the present invention, blood from a donor is drawn and monocytes, naive T cells and naive B cells are separated therefrom. The human whole blood can be collected in heparin containing tubes. Although this is the preferred method of obtaining whole blood, any other method, such as using a needle and heparin, ACD, Citrate or EDTA coated syringe, is acceptable. The monocytes and peripheral blood lymphocytes can be separated using a density gradient such as Ficoll-Hypaque™ (Pharmacia Biotechnology Group, Uppsala, Sweden). Other methods, including magnetic bead assisted separation (MACs and Dynel technologies that are capable of separating the desired components from the rest of the components of the whole blood are also acceptable. Notably, at this time the separation products including the monocytes, naïve T cells and naïve B-cells can be frozen for later use due to the culturing method of the present invention wherein the monocytes are matured first and in a culturing medium that does not include T-cells or B-cells.

Once the monocytes are separated from the PBL, the monocytes are matured into monocytes derived dendritic cells with any agent that promotes monocyte maturation. Preferably, the monocytes are differentiated into monocyte derived dendritic cells (MDCs) by culturing for 4 to 7 days in the presence of activation agents. For example, stem-cell-derived- or monocyte-derived DCs can be sustained ex vivo with GM-CSF and other cytokines and can be matured in vitro by bacteria, viruses, fungi, bacterial products, such as lipopolysaccharide (LPS), inflammatory stimuli, and cytokines, including interferons, interleukin-1 (IL-1), tumor necrosis factor alpha (TNF-α) and its superfamily, RANTES and most often, by CD40 ligand, which plays an important role in DC/T-cell interaction. Specifically, the monocyte maturation-promoting agent may be any compound which facilitates the development and differentiation of monocytes to dendritic cells. Suitable monocyte maturation-promoting agents include, but not limited to the following agents IL-1, GM-CSF, IL-3, IL-4, IL-6, TNF-α, G-CSF, M-CSF, IL-12, IL-15, IL-18 or mixture thereof. Preferably, the monocyte maturation-promoting agent is GM-CSF alone or in combination with an additional maturation agent such as IL-4(4). Further any additional component that increases the rate of monocyte maturation may also be included, such as histamine. Optimal conditions for culturing should be considered including temperature, humidity, pH and the addition of carbon dioxide with a timeframe ranging from about 4 to 10 days.

Maturation stimulates increased expression of HLA-DR, CD40, and costimulatory molecules and secretion of cytokines which is important because MDCs are the sole population of antigen presenting cells in vivo that initiate primary immune responses. Notably, it should be recognized that yields and types of expressed receptors can be influenced by culture components. In the present invention, the T-cells and B-cells, isolated from the same sample as that of the monocytes are not included in the culturing medium for the differentiation of the monocytes.

Once the monocytes have matured into monocyte dendritic cells, they are isolated from the culture medium and combined with naive T-cells and naïve B-cells, all cells preferably from a single donor, and a target antigen for production of antigen specific B-Cells and antibodies raised against the target antigen.

Additionally an adjuvant or combination of adjuvants may be included in the culture medium to enhance the immune response including, but not limited to, the A subunit of cholera toxin or parts thereof (e.g. the Al domain of the A subunit of Ctx from any classical Vibrio cholerae or El Tor V. cholerae strain. Alternatively, any bacterial toxin that increases cellular cAMP levels, such as a member of the family of bacterial adenosine diphosphate-ribosylating exotoxins may be used in place of CtxA, for example the A subunit of heat-labile toxin (referred to herein as EltA) of enterotoxigenic Escherichia coli, pertussis toxin S1 subunit; as a further alternative the adjuvant may be one of the adenylate cyclase-hemolysins of Bordetella pertussis, Bordetella bronchiseptica or Bordetella parapertussis, B. parapertussis or B. bronchiseptica.

Other adjuvants that may be used in the present invention include cytokines, such as IL-4, IL-5, IL-6, IL-10, 11-12, 11-18, TGF or M60316, IFN-γ and TNFα or chemokines, such as MIP-1α, MIP-1β, MIP3α, MDC, RANTES, IL-8, and SDF-1α. Notably, the adjuvant may be chosen to provide for not only the production of primary antibodies IgM but also other types such as IgG and IgA.

The antibodies generated by this method are polyclonal and can be separated by methods known to those skilled in the art including ELISA or other binding affinity assays. Thus, the method and system of the present invention provides for evaluating potential vaccines for human use.

In addition, the method and system of the present invention can be used to generate human monoclonal antibodies on demand. This can be accomplished by immortalization of the antigen specific B cells with Epstein Bar Virus (EBV)(6-8) or by conventional hybridoma methods using a human fusion partner (9-11). Both of these methods have been used to develop human monoclonal antibodies but they are inefficient and require that the B cell donor be immune to the test antigen. Immunization in vitro prior to B cell immortalization via EBV or cell fusion obviates the need to identify immune cell donors or to deliberately immunize a cell donor with the test vaccine.

Thus, the method and system of the present invention circumvents the problem of having to identify immune individuals as a source of B cells to generate monoclonal antibodies. This is particularly important in situations when therapeutic antibodies are desired where it is difficult (or not possible) to deliberately immunize an individual and the target disease is rare. There are many such situations in the biodefense arena, ricin and botulinum toxin are two important examples.

The antibody producing B cells generated by the system of the present invention are suitable for fusion with a myeloma line for the ultimate production of monoclonal antibodies. Specialized myeloma cell lines have been developed from lymphocyte tumors for use in hybridoma-producing fusion procedures (12, 13). It is preferred that human myeloma cells are used in the fusion procedure. The myeloma cells are introduced into the system with the inclusion of an agent that promotes the formation of the fused myeloma and B-cells, such as polyethylene glycol (PEG) and Dimethyl sulfoxide (DMSO). Alternatively, fusion can be induced by electrofusion or via fusiogenic viruses such as Sendai virus.

Methods for generating hybrids of antibody-producing B-cells and myeloma cells usually comprise mixing B cells with myeloma cells in a 2:1 proportion (though the proportion may vary from about 20:1 to about 1:1), respectively, in the presence of an agent or agents that promote the fusion of cell membranes. Fusion procedures usually produce viable hybrids at very low frequency and as such, it is essential to have a means of selecting the fused cell hybrids from the remaining unfused cells, particularly the unfused myeloma cells. A means of detecting the desired antibody-producing hybridomas among other resulting fused cell hybrids is also necessary.

Generally, the selection of fused cell hybrids is accomplished by culturing the cells in media that support the growth of hybridomas but prevent the growth of the myeloma cells which normally would go on dividing indefinitely. (The B-cells used in the fusion do not maintain viability in in vitro culture and hence do not pose a problem.) Generally, the myeloma cells used in the fusion lack hypoxanthine phosphoribosyl transferase. These cells are selected against in hypoxanthine/aminopterin/thymidine (HAT) medium, a medium in which the fused cell hybrids survive due to the HPRT-positive genotype of the spleen cells. The use of myeloma cells with different genetic deficiencies (e.g., other enzyme deficiencies, drug sensitivities, etc.) that can be selected against in media supporting the growth of genotypically competent hybrids is also possible.

Several weeks are required to selectively culture the fused cell hybrids. Early in this time period, it is necessary to identify those hybrids which produce the desired antibody so that they may be subsequently cloned and propagated. The detection of antibody-producing hybrids can be achieved by any one of several standard assay methods, including enzyme-linked immunoassay and radioimmunoassay techniques which have been described in the literature.

Once the desired fused cell hybrids have been selected and cloned into individual antibody-producing cell lines, each cell line may be propagated in vitro in laboratory culture vessels; the culture medium, also containing high concentrations of a single specific monoclonal antibody, can be harvested by decantation, filtration or centrifugation

In the alternative, the antibody producing B-cells that are generated by the system of the present invention, can be suspended in EBV infected culture supernatant and incubated. The EBV infected B-cells are immortalizes upon infection. Notably the EBV infected culture may be introduced at the same time as the naive B cell or subsequent to formation of antibody producing B cells. Also, these lymphocytes may be fused to an appropriate fusion partner in order to produce a stable, monoclonal producing hybridoma.

The method of the invention can be used analytically as described above in connection with immune deficiencies. The ability of the present invention to use specific antigens for the sensitization of human B cells makes it now possible to produce monoclonal human antibodies of defined specificity which can be used to diagnostic and therapeutic advantage.

The following examples describe the new inventive method. These examples are given merely for illustration of the present invention and are not to be construed as a limitation on the remainder of the specification in any way.

### Example 1

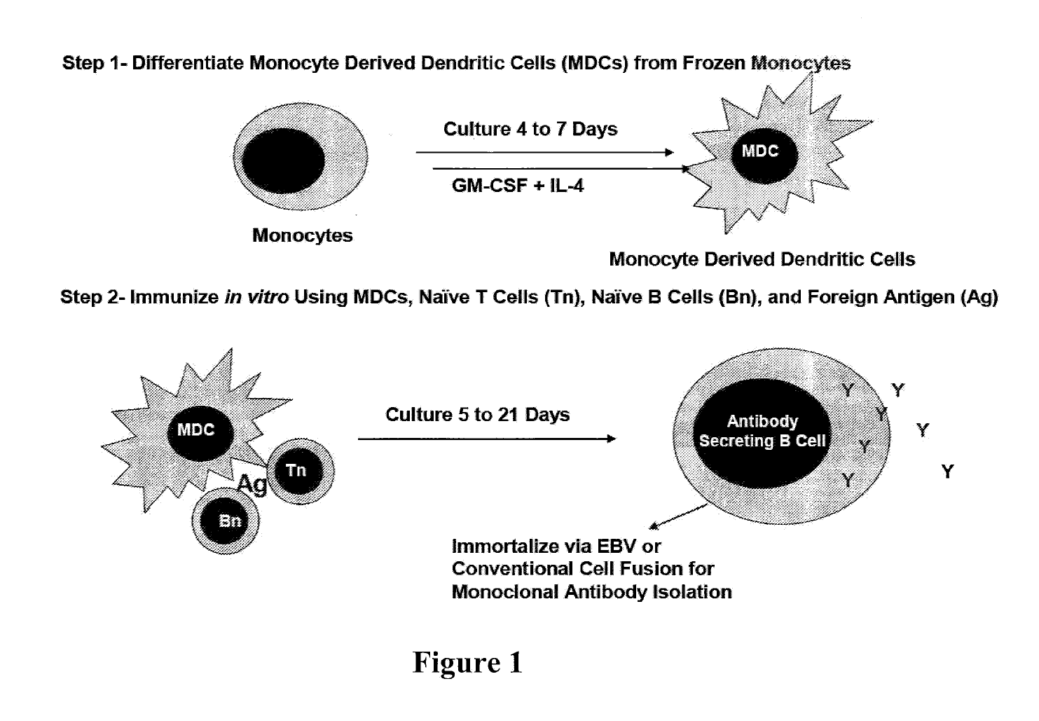

The system is diagrammed in FIG. 1. In this system, peripheral blood is obtained from normal human volunteers and three populations of hematopoetic cells are isolated and frozen as a source of tissues for generating the antibody responses. These three populations are naïve CD4+ T cells (Tn, defined as CD4+ CD45R0− and CD62L+), naïve B cells (Bn, defined as CD19+CD27−), and monocytes (defined as CD14+ CD83−). These cells may be stored frozen in liquid nitrogen to provide a uniform source of cells for immunization in vitro. If desired, it is also possible to define these cells genetically to determine the relationships between immune responses and allelic variants of genes that control these responses.

As shown in FIG. 1, the system requires two phases of culture to generate an antibody response in vitro. Initially, isolated monocytes are differentiated into monocyte derived dendritic cells (MDCs) by culturing for 4-7 in the presence of an activating agent, such as GM-CSF and IL-4 (4).

Dendritic cells (DC) from monocyte preparations can be produced by immunomagnetic CD14+ selection using a semiautomated clinical scale immunomagnetic column (4). The immunomagnetic enrichment of CD14+ monocytes using antibodies linked to ferromagnetic beads can generate pure preparations of monocytes. A clinical scale immunomagnetic column (CliniMACS, Miltenyi Biotec, Bergisch Gladbach, Germany) has become available for the positive selection of monocytes for in vitro use of the MDC populations. With the immunomagnetic separation, a cell suspension of high CD14+ purity (median 97.4%, range 94.9-99.0) with a high monocyte yield (median 82.3%, range 63.9-100.0) can be achieved. Thus, using immunomagnetic isolation of CD14+ monocytes with the CliniMACS® device is a suitable method for clinical-scale generation of MDC.

Differentiation of COD+ cells into mature monocyte-derived DC can be induced by incubation with IL-4, GM-CSV and/or in combination with TEN-α, PEG, IL-1β, and IL-6. For example, the selected CD14+ cells can be incubated at 37° C. in 5% CO2 in culture flasks in X-Vivo 15 medium and supplemented with IL-4 and GM-CSF to induce maturation. Monocytes can be differentiated in vitro into mature DCs as indicated by decreasing CD14+ expression, increasing CD83 expression and upregulation of the co-stimulatory molecules CD80 and CD86. Mature DC include expression of CD83, HLA-DR, and the co-stimulatory molecules CD80 and CD86.

The MDCs are then cultured in vitro with naïve T and B cells from the same donor with the target antigen for about 4 to 21 days to generate a primary antibody response. T cells are required as a source of “help” for T dependent antigens. The culture medium may include RPMI-1640, Dulbecco's Modified Eagles' Media, or Iscove's Modified Dulbecco's Media, a lymphokine(s) capable of inducing proliferation and differentiation of T and B cells. The lymphokines useful in the practice of the subject invention will be apparent to those skilled in the art and include IL-1 and IL-2, B-cell growth factor, B-cell differentiation factor, interferon, colony-stimulating factor, thymic hormones, maturation factor and epidermal growth factor. A candidate adjuvant can be included in the culture to assess its ability to augment the antibody response. Antibody responses can be quantified by any of a variety of methods including, ELISA, ELISPOT, or direct antigen binding to the responding B cells (using fluorescent antigens).

### Example 2

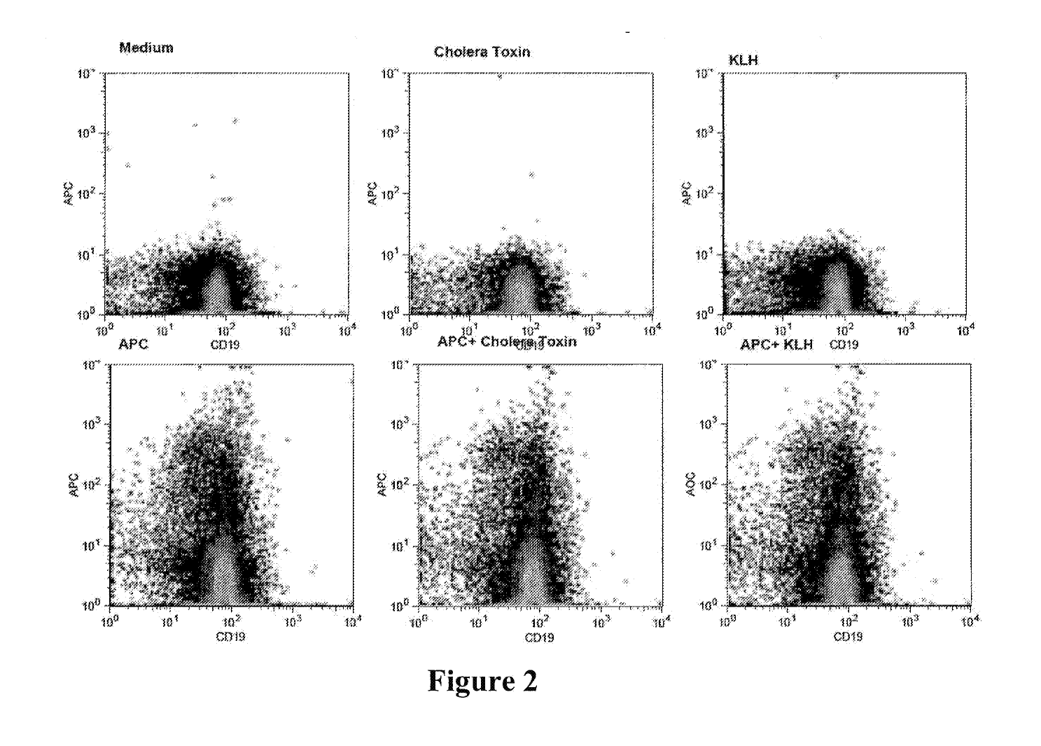

In addition, it is possible to simultaneously quantify MDC activation and the antigen specific CD4+ T cell response by changes in surface marker expression and, in the case of CD4+ T cells, by cell division monitored via the dilution of a vital dye such as carboxyfluorescein-succinimide ester. Thus, this system can be used to measure responses in all of the cell populations relevant to the generation of primary human antibody responses. An example response to the highly fluorescent protein, allophycocyanins (APC), from cyanobacteria is shown in FIG. 2. In this study, cultures comprised of MDCs, naïve T cells, and naïve B cells from the same donor were cultured with either medium alone (5) or the test antigen APC either alone (APC) or with additional antigens giant keyhole limpet hemocyanin (KLH) and cholera toxin (CT). CT is also a powerful adjuvant for antibody responses. On day 5 the B cells (shown as CD19+ in FIG. 2) were analyzed by flow cytometry for their ability to bind APC. As shown in FIG. 2, APC binding B cells (B cells are CD19+ and depicted on the x axis) were detected only in cultures immunized with APC, indicating the induction of a specific antibody response to this immunogen. Thus, this system can be used to generate and quantify primary antibody responses in humans.

### REFERENCES

The contents of all references cited herein are hereby incorporated by reference herein for all purposes.
1. Reichert, J. M., and M. C. Dewitz. 2006. Anti-infective monoclonal antibodies: perils and promise of development. Nat Rev Drug Discov 5:191-195.
2. Reichert, J. M., C. J. Rosensweig, L. B. Faden, and M. C. Dewitz. 2005. Monoclonal antibody successes in the clinic. Nat Biotechnol 23:1073-1078.
3. Vinuesa, C. G., S. G. Tangye, B. Moser, and C. R. Mackay. 2005. Follicular B helper T cells in antibody responses and autoimmunity. Nat Rev Immunol 5:853-865.
4. Babatz, J., C. Rollig, U. Oelschlagel, S. Zhao, G. Ehninger, M. Schmitz, and M. Bornhauser. 2003. Large-scale immunomagnetic selection of CD14+ monocytes to generate dendritic cells for cancer immunotherapy: a phase I study. J Hematother Stem Cell Res 12:515-523.
5. Cohen-Bazire, G., S. Beguin, S. Rimon, A. N. Glazer, and D. M. Brown. 1977. Physico-chemical and immunological properties of allophycocyanins. Arch Microbiol 111:225-238.
6. Steinitz, M., S. Koskimies, G. Klein, and O. Makela. 1978. Establishment of specific antibody producing human lines by antigen preselection and EBV transformation. Curr Top Microbiol Immunol 81:156-163.
7. Kozbor, D., M. Steinitz, G. Klein, S. Koskimies, and O. Makela. 1979. Establishment of anti-TNP antibody-producing human lymphoid lines by preselection for hapten binding followed by EBV transformation. Scand J Immunol 10:187-194.
8. Steinitz, M., S. Koskimies, G. Klein, and O. Makela. 1979. Establishment of specific antibody producing human lines by antigen preselection and Epstein-Barr virus (EBV)-transformation. J Clin Lab Immunol 2:1-7.
9. Croce, C. M., A. Linnenbach, W. Hall, Z. Steplewski, and H. Koprowski. 1980. Production of human hybridomas secreting antibodies to measles virus. Nature 288:488-489.
10. Olsson, L., and H. S. Kaplan. 1980. Human-human hybridomas producing monoclonal antibodies of predefined antigenic specificity. Proc Natl Acad Sci USA 77:5429-5431.
11. Olsson, L., and H. S. Kaplan. 1983. Human-human monoclonal antibodyproducing hybridomas: technical aspects. Methods Enzymol 92:3-16.
12. Kohler G and C. Milstein, 1976. Europe. J. Immunol. 6: 511-519.
13. Shulman, M et al., 1978. Nature 276: 269-270.

That which is claimed is:

1. A two phase culturing method for generating antibodies specific for a target antigen and antigen-specific B-cells, the method comprising:
removing a blood sample from a donor;
isolating naive T cells, naive B cells and monocytes from the sample into three separate populations;
culturing the monocytes in a first culture medium for differentiating the monocytes into monocyte derived dendritic cells (MDC);
isolating the monocyte derived dendritic cells from the first culture medium;
culturing the monocytes derived dendritic cells with the naïve T cell, naïve B cells and the target antigen in a second culture medium; and
isolating recovered generated antibodies and/or antigen-specific B-cells.

2. The two phase culturing method of claim 1, wherein the naive T cells, naive B cells and monocytes can be frozen before use.

3. The two phase culturing method of claim 1, wherein the first culturing medium comprising GM-CSF and IL-4.

4. The two phase culturing method of claim 1, wherein the monocytes are retained in the first culture medium for about 4 to 7 days.

5. The two phase culturing method of claim 3, wherein the MDC include CD14− and CD83+/−.

6. The two phase culturing method of claim 5 wherein the MDC and naïve T cells and B cells are cultured in the second medium for about 4 to 21 days.

7. The two phase culturing method of claim 6 wherein the second culture medium comprises RPMI-1640, Dulbecco's Modified Eagles' Media, or Iscove's Modified Dulbecco's Media.

8. The two phase culturing method of claim 1, wherein the second culture medium further comprises an adjuvant.

9. The two phase culturing method of claim 1, wherein the monocytes include CD14+ and CD 83−.

10. A method for testing immunogen/adjuvant combinations to determine optimal level of antibody generation and/or most effective adjuvant; the method comprising:
removing a blood sample from a donor;
isolating naive T cells, naive B cells and monocytes from the sample into three separate populations;
culturing the monocytes in a first culture medium for differentiating the monocytes into monocyte derived dendritic cells (MDC);
isolating the monocyte derived dendritic cells from the first culture medium;
culturing the monocytes derived dendritic cells with the naïve T cell, naïve B cells and the target antigen in a second culture medium;
adding an adjuvant to the culture medium; and
isolating recovered generated antibodies and determining level of immune response relative to a system without the addition of the adjuvant.

11. The method of claim 10, wherein the first culturing medium comprising GM-CSF and IL-4.

12. The method of claim 10, wherein the monocytes are retained in the first culture medium for about 4 to 7 days.

13. The method of claim 10, wherein the MDC include CD14− and CD83+/−.

14. The method of claim 10 wherein the MDC and naïve T cells and B cells are cultured in the second medium for about 4 to 21 days.

15. The method of claim 10, wherein the monocytes include CD14+ and CD 83−.

16. The method of claim 10, wherein the monocytes, naïve T cells and B cells are kept frozen until use.

17. An in vitro production of monoclonal antibodies without the need of a cell donor immune to the test antigen, the method comprising:
removing a blood sample from a donor;
isolating at least naive T cells, naive B cells and monocytes from the sample;
differentiating the monocytes into monocyte derived dendritic cells;
culturing the monocytes derived dendritic cells with T cell, B cells and the target antigen to generate antibody producing B cells;
isolating the antibody producing B cells;
infecting the antibody producing B cells with Epstein Barr virus and/or fusing the antibody producing B cells with an active myeloma cell to form hybridoma cells;
culturing the hybridoma cells under suitable conditions for expressing desired antibodies; and
recovering the hybridoma cells that generate the desired monoclonal antibody and cloning same.

18. A culture system for in vitro production of antibodies, the system comprising:
(a) a first culture medium comprising monocytes isolated from sample and a activating agent for differentiating the monocyte into monocyte derived dendritic cells (MDCs); and
(b) a second culture medium comprising the MDC isolated from the first culture medium, naïve T cells and B cells isolated from the same sample as (a), a target antigen, and culturing components for the induction of antibodies.

19. The culture system of claim 18, wherein the first culture medium comprises GM-CSF and IL-4 as activating agents.

20. The culture system of claim 18, wherein the MDC include CD14− and CD83+/−.

21. The culture system of claim 18, wherein the second culture medium comprises RPMI-1640, Dulbecco's Modified Eagles' Media, or Iscove's Modified Dulbecco's Media.

22. The culture system of claim 18, wherein the second culture medium further comprises an adjuvant.

23. The culture system of claim 18, wherein the monocytes include CD14+ and CD 83−.
